(19) United States
(12) Patent Application Publication
(10) Pub. No.: US 2008/0311129 A1
(43) Pub. Date: Dec. 18, 2008

(54) ADHERENCE INHIBITOR DIRECTED TO AND METHOD OF MAKING AND USING

(75) Inventors: Peter Nash, Eden Prairie, MN (US); Bradley M. Mitteness, Ghent, MN (US); Michael O'Rourke, New Prague, MN (US)

Correspondence Address: WESTMAN CHAMPLIN & KELLY, P.A., SUITE 1400, 900 SECOND AVENUE SOUTH, MINNEAPOLIS, MN, 55402-3244, US

(73) Assignee: Camas Incorporated, LeCenter, MN (US)

(21) Appl. No.: 11/933,778

(22) Filed: Nov. 1, 2007

Related U.S. Application Data
(60) Provisional application No. 60/856,096, filed on Nov. 2, 2006.
(60) Provisional application No. 60/984,032, filed on Oct. 31, 2007.

(51) Int. Cl. A61K 39/395; A61P 31/04

(52) U.S. Cl. 424/165.1

(57) ABSTRACT

A method for the production of antibodies or microbial adherents administers is disclosed. The antibodies or microbial adherents inhibitors are administered to a patient suffering from strep throat to substantially prevent the adherents of colony-forming immunogens or haptens in the throat of the patient. Application of the antibodies should decrease the colonization of the oropharnyx, and therefore decrease symptoms of the sore inflamed throat and ultimately decrease the need for antibiotics.

### CROSS-REFERENCE TO RELATED APPLICATION

The present application is based on and claims the benefit of U.S. provisional patent application Ser. No. 60/856,096, filed Nov. 2, 2006, the content of which is hereby incorporated by reference in its entirety.

### BACKGROUND OF THE INVENTION

Streptococcus is a genus of spherical, Gram-positive bacteria that are known to be the primary cause of throat infections. Streptococcal sore throat, or strep throat as it is more commonly called, is an infection of the mucous membranes lining the pharynx. Sometimes the tonsils are also infected (tonsillitis).

Streptococcus is also a concern due to the development of not only throat infections, but also due to the other sequella of the infection, namely rheumatoid heart disease. This is where the organism invades the blood stream and causes vegetations on the heart valves. Streptococcus is one of the most frequent causes for visits to the doctor's office in the United States. It is also one of the most frequent reasons for antibiotic use.

The number of people with sore throats in the United States that seek medical attention is an estimated 120 million visits per year. Approximately 12 to 25 percent of those individuals have positive strep cultures. Far more individuals than those diagnosed with strep throat are placed on antibiotics because there is no other available treatments.

The large numbers of people that are prescribed antibiotics for this illness helps to increase the incidence of resistant bacteria as observed in rising levels of antibiotic resistant infections in the public. Antibiotics that were once almost universally effective against these infections are now approximately 70 to 80% effective. This trend is more prevalent in certain parts of the country and continued increases are expected due to widespread use of antibiotics to treat these very common infections.

It has been discovered that use of antibodies that specifically attack the microorganisms responsible for strep throat are useful in reducing the progression of this illness. Accordingly, the present invention is directed to microbial adherence inhibitors or antibodies for substantially preventing the attachment or adherence of colony-forming immunogens or haptens of Streptococcus that are present during the initiation and or progression of strep throat.

A principal objective of the present invention is to substantially prevent the colonization of Streptococcus species, as well as the growth of such organisms resulting in their substantial elimination from an individual.

Haptens are partial or incomplete immunogens such as certain toxins, which cannot by themselves cause antibody formation but are capable of combining with specific antibodies. Such haptens may include bacterial toxin, yeast mold toxin, viruses, parasite toxins, algae toxins, etc.

Members of the Streptococcus genus are responsible for the occurrence of strep throat, meningitis, bacterial pneumonia, endocarditis (inflammation of the inner layer of the heart), erysipelas (bacterial skin infection) even necrotizing, and fascitis (a rare infection of the deeper layers of skin and subcutaneous tissue commonly known as flesh-eating bacteria infection).

Individual species of this genus of interest in this application include the following S. agalactiae, S. bovis, S. mutans, S. pneumoniae, S. pyogenes, S. sanguinis, S. suis, and S. viridans.

Streptococcus species have been characterized into groups identified by “A” to “O”. Groups A and B (also known as “Group A strep” and “Group B strep”) are the most important based on causing common diseases in humans.

S. pyogenes is the causative agent in Group A for Streptococcal infections including strep throat, acute rheumatic fever, scarlet fever and acute glomerulonephritis (inflation of the glomeruli). Other Streptococcus species may also possess the Group A antigen.

S. agalactiae is a primary causative agent in Group B Streptococcal infections and causes meningitis and neonates in the elderly with occasional systemic bacteremia. S. agalactiae can also colonize in the female reproductive tract. For females who are pregnant, antibiotics are not recommended due to the risk benefit. Therefore this infection goes untreated.

Group D strains of Streptococcus include S. bovis and S. suis. S. bovis is occasionally found to be the causative agent in cases of human endocarditis and also sometimes a rare cause of neonatal septicemia and meningitis. S. suis has also been found to cause infection in humans resulting in meningitis and endocarditis.

S. pneumoniae is a leading cause of bacterial pneumonia and meningitis. S. agalactiae can develop neonatal sepsis, neonatal meningitis and neonatal pneumonia. S. mutans plays a major role in tooth decay, metabolizing sucrose to produce lactic acid and is equipped with receptors for adhesion to the surface of teeth.

S. sanguinis typically found in dental plaque may gain entrance to the blood stream and colonize in heart valves where it is the most common cause of bacterial endocarditis.

In the past, conventional treatments included the use of antibiotics that reduce symptoms, minimize transmission, and reduce the likelihood of complications. Of particular interest, are the Streptococcus species that result in strep throat. Treatment of strep throat generally consists of oral or intramuscular injection of penicillin. Erythromycin is often recommended for penicillin-allergic patients. In addition, other antibiotics, such as amoxicillin, clindamycin, and oral cephalosporins may also be recommended. Although symptoms typically subside within 4 days even without treatment, it is very important to start treatment within 10 days of onset of symptoms, and to complete the full course of antibiotics to prevent rheumatic fever.

As with any use of antibiotics, a major concern is development of antibiotic resistant strains. In addition use of broad-spectrum antibiotics has further drawbacks including vulnerability to human error, additional cost and the like.

In one aspect, the present invention is directed to a method for the production of antibodies or microbial adherence inhibitors for administration to a patient suffering from strep throat to substantially prevent the adherence of colony-forming immunogens or haptens in the throat of the patient. Application of the antibodies should decrease the colonization of the oropharynx, and therefore decrease symptoms of the sore inflamed throat and ultimately decrease the need for antibiotics.

The method includes first inoculating female birds, in or about to reach their egg laying age, with the particular target immunogen derived from Streptococcus. Then after a period of time sufficient to permit the production in the bird of antibody to the targeted immunogen, the eggs laid by the birds are harvested. The total antibody-containing contents of the eggs are separated from the shells and can be dried or used as a liquid. The egg contents may be dried on a carrier material or mixed with other additives or further purified. The dried or liquid separated egg adherence inhibiting material may be stored or shipped for use when needed.

Although application in individuals with strep throat is specifically discussed, the present invention is also applicable to development of other antibodies that can be used for the treatment of other types of upper respiratory and lower respiratory illnesses and illness caused by other species of Streptococcus.

The antibodies of the present invention are designed to target the adhesion molecules of Streptococcus, and therefore will decrease their ability to adhere to the membranes in the oropharynx and decrease their ability to cause an infection in the area.

The antibodies of the present invention will be administered by any conventional method, such as orally, by injection, by inhalation or the like in a manner that permits substantially uniform distribution of the antibodies throughout the target area or the throat of the patient.

The substantial prevention of colonization of a Streptococcus species will ultimately permit substantial reduction or elimination of the organism from the system. In addition, the antibodies may be administered only once, or administered over some substantial period of the illness and even after the symptoms have subsided. In addition, it is expected that administration will vary depending on the individual and severity of the illness.

The Streptococcus species mentioned herein possess the capability of adhering to the affected membrane or organ in order to multiply and grow. In the case of strep throat it is an individuals' throat or pharynx. The antibodies of the present invention are therefore believed capable of strongly interfering with adherence in a highly specific manner and, on a cumulative basis, to thereby prevent the targeted organism from multiplying, growing, and colonizing.

The most successfully colonizing microorganisms, bacteria, viruses, and parasites, etc., have evolved a number of different types of molecules, referred to as “adherens,” on their surfaces which can very tightly stick to one or more molecules that are part of the host's various surfaces. The antibody of the present inventions are capable of high specific activity which can very tightly bind to coat, cover, and obliterate these “adherens” which attach themselves to their host with a lock and key type of fit to very unique chemical structures.

The evidence seen in the animal models of the effectiveness of these antibodies in clearing infection is encouraging. In addition, the science behind these antibodies indicates that it will be very difficult for bacteria known to cause this illness to develop resistance to this approach. This will improve the effectiveness of this method of fighting the bacteria.

The data that has been developed suggests that this approach will clear the bacteria more quickly than conventional antibiotics. This will decrease the incidence of seguelae from the illness.

The cost of this product will allow pricing in a way that will make it unnecessary for a physician to perform a strep test. If this product can be sold for the same price as a strep test costs and there is little chance that a bacteria can develop resistance to the product then there is no need to perform a strep test. With this model a physician will merely prescribe this medicine to all people with a sore throat and then treat those that fail clinically after the medicine is taken. This will greatly reduce the use of antibiotics for this illness. In addition the data that we have from animal models suggest that this will improve clinical symptoms of the disease more rapidly than antibiotics, this will make the patients happy about the use of this product in place of antibiotics.

Marketing the product in this fashion will increase the patient population that will be treated with this product to the entire 120 million people that visited their doctor for sore throats last year. This will also decrease the use of antibiotics in the 50 million people that were given antibiotics for sore throats last year. This will also decrease the cost to the insurance companies. At this time they are paying for the office visit, the strep test and the antibiotics. The increase in resistance to antibiotics forces the insurance company to pay for an additional visit and a second antibiotic. With implementation of this treatment, great reductions in cost should be attained.

1. A method of production of an adherence inhibitor for administration to humans to control the colonization of Streptococcus bacteria by preventing the adherence of the Streptococcus bacteria to a surface of a membrane or organ within the human, the method comprises:
a. inoculating female birds, in or about to reach their egg laying age, with a Streptococcus bacteria;
b. allowing a period of time sufficient to permit the production in the birds of antibody to the Streptococcus bacteria;
C. harvesting the eggs laid by the birds; and
d. separating the antibody-containing contents of said eggs from the shells.

2. The method of claim 1 wherein:
the Streptococcus bacteria includes S. agalactiae, S. bovis, S. mutans, S. pneumoniae, S. pyogenes, S. sanguinis, S. suis, and S. viridans.

3. The method of claim 1 including:
drying the separated antibody-containing contents of said eggs.

4. The method of claim 3 wherein:
the Streptococcus bacteria includes S. agalactiae, S. bovis, S. mutans, S. pneumoniae, S. pyogenes, S. sanguinis, S. suis, and S. viridans.

5. The method of claim 3 including:
providing a dried material, said drying of the separated antibody-containing contents of said eggs is achieved by drying the separated antibody-containing contents of said eggs.

6 The method of claim 5 wherein:
the material is a liquid product achieved by using separated antibody-containing contents of said eggs.

7. A treatment of a Streptococcus bacterial infection, the treatment comprising:
administering an antibody specific to an adheren of the Streptococcus bacteria that causes adhesion to a membrane or organ.

8. The treatment of claim 7 wherein the antibody specific to the adheren of the Streptococcus bacteria is produced by:
a. inoculating female birds, in or about to reach their egg laying age, with a Streptococcus bacteria;
b. allowing a period of time sufficient to permit the production in the birds of antibody to the Streptococcus bacteria;
C. harvesting the eggs laid by the birds; and
d. separating the antibody-containing contents of said eggs from the shells.

9. The treatment of claim 7 wherein the Streptococcus bacteria includes S. agalactiae, S. bovis, S. mutans, S. pneumoniae, S. pyogenes, S. sanguinis, S. suis, and S. viridans.

10. The treatment of claim 8 further including drying the separated antibody-containing contents of said eggs.

11. The treatment of claim 10 wherein the Streptococcus bacteria includes S. agalactiae, S. bovis, S. mutans, S. pneumoniae, S. pyogenes, S. sanguinis, S. suis, and S. viridans

12. A method for substantially reducing or eliminating the incidents of Streptococcus bacteria by inhibiting the ability of the Streptococcus bacteria to adhere to a surface of a membrane or organ thereby reducing the ability of the Streptococcus bacteria to colonize and multiply, the method comprising:
administering an antibody specific to an adheren of the Streptococcus bacteria that causes adhesion to a membrane or organ.

13. The treatment of claim 12 wherein the antibody specific to the adheren of the Streptococcus bacteria is produced by;
a. inoculating female birds, in or about to reach their egg laying age, with a Streptococcus bacteria;
b. allowing a period of time sufficient to permit the production in the birds of antibody to the Streptococcus bacteria;
c. harvesting the eggs laid by the birds; and
d. separating the antibody-containing contents of said eggs from the shells.

14. The treatment of claim 12 wherein the Streptococcus bacteria includes S. agalactiae, S. bovis, S. mutans, S. pneumoniae, S. pyogenes, S. sanguinis, S. suis, and S. viridans.

15. The treatment of claim 13 further including drying the separated antibody-containing contents of said eggs.

16. The treatment of claim 15 wherein the Streptococcus bacteria includes S. agalactiae, S. bovis, S. mutans, S. pneumoniae, S. pyogenes, S. sanguinis, S. suis, and S. viridans
